(19) United States
(12) Patent Application Publication
(10) Pub. No.: US 2008/0181848 A1
(43) Pub. Date: Jul. 31, 2008

(54) METHOD TO DETECT DOPAMINE RECEPTORS IN THE FUNCTIONAL D2HIGH STATE

(76) Inventor: Philip Seeman, Toronto (CA)

Correspondence Address: BERESKIN AND PARR, 40 KING STREET WEST, BOX 401, TORONTO, ON, M5H 3Y2, omitted

(21) Appl. No.: 11/948,310

(22) Filed: Nov. 30, 2007

Related U.S. Application Data
(63) Continuation-in-part of application No. 11/558,649, filed on Nov. 10, 2006.
Application No. 11/558,649 is a continuation-in-part of application No. PCT/CA2005/001662, filed on Nov. 2, 2005.
(60) Provisional application No. 60/624,192, filed on Nov. 3, 2004.
(60) Provisional application No. 60/687,443, filed on Jun. 6, 2005.

(51) Int. Cl. A61K 51/04; G01N 33/566

(52) U.S. Cl. 424/1.81; 424/1.65; 436/501

(57) ABSTRACT

This application describes a method for identifying dopamine supersensitivity using radioactive (+)PHNO. The method involves determining the specific binding of radioactive (+)PHNO in the subject's brain. An increase in the specific binding of radioactive (+)PHNO in the subject compared to a control indicates that the subject is in a state of dopamine supersensitivity. The extent of dopamine supersensitivity can be used to assess, treat and/or follow the progress of any dopamine-related disorder.

### FIELD OF THE INVENTION

The present invention relates to methods for detecting, observing and determining the amount of dopamine D2^{high} receptors in a subject's brain. The amount of dopamine D2^{high} receptors in a subject's brain may be used, for example, for detecting and diagnosing supersensitivity of the dopamine neurotransmission system in the human brain in health and disease, including psychosis, schizophrenia, addiction, attention-deficit hyperactivity disorder (ADHD) and Parkinson's disease.

### BACKGROUND OF THE INVENTION

Psychotic symptoms occur in many diseases, including schizophrenia and prolonged drug abuse. Although many chromosome regions and genes have been found associated with schizophrenia, no single gene of major effect has yet been identified. Nevertheless, regardless of the causes of psychosis, antipsychotic drugs are mostly effective in alleviating the symptoms. Because the clinical antipsychotic potencies of these drugs are directly related to their affinities for the dopamine D2 receptor (P. Seeman, M. Chau-Wong, M. Tedesco, K. Wong, Proc. Nat. Acad. Sci, U.S.A. 72: 4376, 1975; P. Seeman, Can. J. Psychiatry 47: 27, 2002), it suggests that the properties of this receptor are disturbed in psychosis. It is uncertain whether the total density of D2 receptors in schizophrenia is elevated (A. L. Nordstrom, L. Farde, L. Eriksson, C. Halldin, Psychiatry Res. 61: 67, 1995; A. Abi-Dargham et al. Proc. Nat. Acad. Sci, U.S.A. 72: 7673, 2000).

The more relevant question, however, is whether the functional state of D2, or the state of high-affinity for dopamine, D2^{High} (S. R. George et al., Endocrinology 117: 690, 1985), is elevated, and this has not been investigated in schizophrenia or in any of the psychoses. There are two classes of dopamine receptors in the brain, type D1 and type D2. The dopamine receptors are part of the general family of G protein-linked receptors. A receptor which is linked to a G protein (of which there are many types) can exist in two states. One state has a high affinity for the neurotransmitter, dopamine, with a dissociation constant of 1.5 nM for the D2 receptor, for example, and this state is referred to as the high-affinity state, or D2^{High}. The other state has a low affinity for the neurotransmitter, dopamine, with a dissociation constant of approximately 200-2000 nM for the D2 receptor, for example, and this state is referred to as the low-affinity state, or D2_{Low}. Depending on local conditions in vitro or in vivo, the two states can quickly convert into each other. Because the high-affinity state is considered the functional state (S. R. George et al., Endocrinology 117: 690, 1985), the process of “desensitization” occurs whenever the high-affinity state converts into the low-affinity state.

An increased number of elevated density of D2^{High} receptors would explain why individuals with schizophrenia or psychosis are supersensitive to dopamine or dopamine-mimetics (J. A. Lieberman, J. M. Kane, J. Alvir, Psychopharmacology 91: 415, 1987).

Normally, the proportion of D2 receptors which are in the high-affinity state, as measured in homogenized rat striatal tissue in vitro, is ˜10-20% (Seeman, et al., Proc. Nat. Acad. Sci., U.S.A., Mar. 1, 2005). This has been determined using the competition between dopamine and [³H]raclopride or [³H]domperidone (Seeman, et al., Proc. Nat. Acad. Sci., U.S.A., Mar. 1, 2005). Several previous studies have examined whether the proportion of high-affinity states for D2 were elevated after antipsychotics or in GRK6 knockouts, with little if any significant change (Seeman, et al., Proc. Nat. Acad. Sci., U.S.A., Mar. 1, 2005). This is because most laboratories have been using a dopamine/[³H]spiperone competition method. As shown elsewhere, however, dopamine (with its Kd of 1.75 nM) is not effective in competing versus the much more tightly bound [³H]spiperone (with its Kd of 60 pM), especially in 120 mM NaCl.

Using three methods, especially using dopamine/[³H]domperidone competition experiments, all types of animals that are supersensitive to dopamine, dopamine-mimetics or amphetamine, were found to have a higher proportion of D2^{High} receptors (Seeman, et al., Proc. Nat. Acad. Sci., U.S.A., Mar. 1, 2005). In fact, it was found that there was a 100% to 900% elevation in the proportion of D2 receptors in the high-affinity state in rats after neonatal hippocampal lesions, in rats after long-term treatment with antipsychotics, ethanol, or amphetamine, and in mice with gene knockouts of Dbh (dopamine β-hydroxylase), D4 receptors, GRK6 (G protein-coupled receptor kinase 6) or COMT (catechol-O-methyltransferase), and in rats born by Caesarian-section.

In principle, the high-affinity state of the D2 receptor can be labelled by low concentrations of various radioactive dopamine agonists, including dopamine, apomorphine, N-propyl-norapomorphine, quinpirole, aminotetralins, and a variety of dopamine-related congeners.

Quinpirole is a dopamine agonist which is often used as a selective agonist for D2 receptors. Although quinpirole has a 250-fold selectivity for dopamine D2 receptors over D1 receptors (P. Seeman and J. M. Schaus, Eur. J. Pharmacol. 203: 105-109, 1991), its high dissociation constant of 5 nM makes it vulnerable to inhibition by endogenous dopamine. Moreover, radioactive quinpirole has high nonspecific binding, indicating that this compound binds to many other unidentified sites.

Although (−)-N—[¹¹C]propyl-norapomorphine has been used to label D2 receptors (R. Narendran et al., Synapse 52: 188-208, 2004), it is known that propyl-norapomorphine has an equal affinity for the D1 and D2 receptors, with a 0.7 nM dissociation constant at both receptors.

A highly selective agonist for the high-affinity state of the D2 receptor is (+)-4-propyl-9-hydroxy-2,3,4a,5,6,10b-hexahydro-4H-naphth[1,2b][1,4]oxazine HCl, or (+)PHNO (also known as (+)-4-propyl-9-hydroxynaphthoxazine, MK458, L-647,339 or naxagolide). Although (+)PHNO is effective in alleviating Parkinson's disease (A. Lieberman et al., Clin. Neuropharmacol. 11: 191-200, 1988), its long-term use may lead to desensitization and a loss of clinical efficacy (J. M. Cedarbaum et al., Movement Disorders 5: 298-303, 1990). The preparation of [³H]PHNO and [¹¹C](+)PHNO have been previously described (P. Seeman, et al. Synapse 14: 254-262, 1993; J. Brown, S. K. Luthra, F. Brady, C. Prenant, D. Dijkstra, H. Wikstrom, and D. Brooks, Labelling of the D2 agonist (+)—PHNO using [¹¹C]-Propionyl Chloride, in XIIth Int. Symp. Radiopharmaceutical Chemistry, pp. 565-566, Uppsala, Sweden, 1997).

Experimentally, dopamine supersensitivity occurs after a neonatal lesion of the brain (S. K. Bhardwaj, et al., Neuroscience 122: 669, 2003); prolonged use of antipsychotics (T. F. Seeger, et al., Psychopharmacology 76: 182, 1982), ethanol or amphetamine (T. E. Robinson, K. C. Berridge, Addiction 95(Suppl. 2): S91, 2000); in gene knockouts of Dbh (dopamine β-hydroxylase) (D. Weinshenker, et al., Proc. Nat. Acad. Sci., USA 99: 13873, 2002), dopamine D4 receptors (M. Rubinstein et al., Cell, 90:1991, 1997), GRK6 (G protein-coupled receptor kinase 6 (R. R. Gainetdinov et al., Neuron 38: 291, 2003), or COMT (catechol-O-methyltransferase) (M. Huotari, et al., Psychopharmacology 172: 1, 2004); and in rats born by C-section (P. Boksa, et al., Exper. Neurol. 175: 388, 2002). While antipsychotics are known to elevate the density of dopamine D2 receptors by ˜25% above control levels, no such elevations occur in ethanol withdrawal (P. Seeman, et al., Synapse 52: 77, 2004), in amphetamine-sensitized animals (P. Seeman, et al. Synapse 46: 235, 2002), in GRK6 or COMT knockouts, or in rats born by C-section.

The basis of supersensitivity to amphetamine or dopamine agonists thus remains puzzling. However, it has recently been found that, despite the absence of any elevation in total dopamine D2 receptors in the striata of amphetamine-sensitized animals, there is a dramatic 360% increase (P. Seeman, et al., Synapse 46, 235, 2002) in the density of D2^{High} states. It has more recently been found that the density or proportion of D2^{High} states is also invariably elevated in other conditions showing dopamine supersensitivity. This was found to be the case in studying the brain striata from many types of animals that are known to be dopamine supersensitive after treatment with either antipsychotics, quinpirole, ethanol or amphetamine, after a hippocampal lesion, or following four types of gene knockouts mentioned above (Seeman, et al., Proc. Nat. Acad. Sci., U.S.A., Mar. 1, 2005).

An alteration in the amount or density of dopamine receptors in the D2^{high} state in specific regions of the brain can be an indication of dopamine-related illnesses. For example, the state of dopamine supersensitivity, correlated with an elevated number of D2^{high} receptors, usually develops in early stages of dopamine-related diseases. In order to assess, treat, and follow the progress of such dopamine-related illnesses, there is a need for methods to measure the amount or density of dopamine receptors in the D2^{high} state.

### SUMMARY OF THE INVENTION

Described herein is a method for identifying and quantitating the amount or density of D2^{high} receptors in the brain in various stages of a dopamine-related disease. The method comprises obtaining radioactive (+)-4-propyl-9-hydroxynaphthoxazine (or (+)PHNO), for example [¹¹C](+)-4-propyl-9-hydroxynaphthoxazine ([¹¹C](+)-PHNO) and injecting a trace amount intravenously into a subject, for example a human, and imaging, for example by means of positron emission tomography, the amount of radioactive (+)PHNO localized to the brain, in particular the striatum, caudate nucleus, putamen regions and the globus pallidus. Several hours later, a second injection of radioactive (+)PHNO is given intravenously contemporaneously with a low dose of a non-radiolabelled dopamine agonist or dopamine mimetic having an affinity or dissociation constant for the dopamine D2 receptor that is similar to the radioactive (+)PHNO, for example between about 0.4 to about 0.9 nM for the high-affinity state of dopamine D2 receptors, and with a permeability across biological membranes that is similar to that for (+)PHNO. The contemporaneously administered dose of dopamine agonist or dopamine mimetic should be on the order of about 10 to about 50 times the dose of total active drug or active ingredient in the radiolabelled (+)PHNO dose (radiolabelled and non-radiolabelled molecules), thus defining a baseline to determine the number of high-affinity states of D2 in the same brain region. The difference between the brain image obtained without contemporaneous administration of non-radiolabelled dopamine agonist or mimetic and the image obtained with contemporaneous injection of non-radioactive dopamine agonist or mimetic is designated as the “specific binding” of radioactive (+)PHNO. The amount of specific binding of radioactive (+)PHNO localized in a particular region of the brain is related to the extent of dopamine sensitivity of the brain, with much higher than normal amounts reflecting the presence of more high-affinity states of D2 receptors with an associated significant dopamine supersensitivity in behaviour and supersensitivity to dopamine agonists.

Accordingly, the present invention relates to a method for determining an amount of dopamine D2^{high} receptors in a subject comprising determining specific binding of radiolabelled (+)-4-propyl-9-hydroxy-2,3,4a,5,6,10b-hexahydro-4H-naphth[1,2b][1,4]-oxazine HCl ((+)PHNO) in the subject's brain. The presence of radiolabelled (+)PHNO in the subject's brain, indicates the presence of dopamine D2^{high} receptors. Further the specific binding of radiolabelled (+)PHNO in the subject's brain is correlated with the amount of dopamine D2^{high} receptors in that area, such that the greater the specific binding of radiolabelled (+)PHNO, the greater the number of D2^{high} receptors. In an embodiment of the present invention, the specific binding of radiolabelled (+)PHNO in the brain of the subject is compared to a control and if the specific binding is greater in the subject compared to the control then the subject is in a state of dopamine supersensitivity.

Accordingly, in a further embodiment of the present invention, there is included a method of determining an extent of dopamine supersensitivity in a subject comprising determining specific binding of radiolabelled (+)PHNO in the subject's brain.

The extent of dopamine supersensitivity is an important factor in the assessment of health and disease in a subject, for example, to assess, treat and/or follow the progress of any dopamine-related disorder.

The present invention also includes the use of radiolabelled (+)PHNO to determine an amount of dopamine D2^{high} receptors in a subject as well as the use of radiolabelled (+)PHNO to prepare a medicament to determine an amount of dopamine D2^{high} receptors in a subject.

Further, the present invention includes the use of radiolabelled (+)PHNO to determine an extent of dopamine supersensitivity in a subject as well as the use of radiolabelled (+)PHNO to prepare a medicament to determine an extent of dopamine supersensitivity in a subject.

In a further embodiment of the invention, the radiolabelled (+)PHNO is [¹¹C]-(+)-4-propyl-9-hydroxy-2,3,4a,5,6,10b-hexahydro-4H-naphth[1,2b][1,4]-oxazine HCl or [¹¹C]-(+)PHNO.

The present invention further includes a method of screening for compounds that bind to the D2^{High} receptor comprising (a) combining a sample comprising the D2^{High} receptor with radiolabelled (+)PHNO and test compound under conditions sufficient for binding of radiolabelled (+)PHNO and the test compound to the D2^{High} receptor; and (b) determining an amount of binding of the radiolabelled (+)PHNO that is inhibited in the presence of the test compound. The invention also includes the use of radiolabelled (+)PHNO to screen for compounds that bind to the D2^{High} receptors.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DETAILED DESCRIPTION OF THE INVENTION

Although there are many molecular mechanisms underlying dopamine supersensitivity, the common component is an increase in the number or proportion of D2 receptors in the high-affinity state for dopamine. The D2 receptor is the primary target for all antipsychotic drugs. There are five types of dopamine receptors in humans (D1, D2, D3, D4, and D5; Neuropsychopharmacology 7: 261-284, 1992). Of these, only the D2 receptor is blocked by antipsychotic drugs in direct relation to their clinical antipsychotic potencies (Nature 261: 717-719, 1976). As stated by Su et al. (Arch. Gen. Psychiat. 54: 972-973, 1997), “ . . . no drug has yet been identified with antipsychotic action without a significant affinity for the dopamine D2 receptor.” Furthermore, the concentrations of antipsychotic drugs that block dopamine D2 receptors in vitro are identical to the concentrations of antipsychotic drugs that are found in the spinal fluid or in the plasma water (i.e., corrected for drug binding to the plasma proteins) of patients being successfully maintained on these drugs (P. Seeman, Canad. J. Psychiat. 47: 27-38, 2002). Therefore, because D2 is the common target for antipsychotic drugs, and because the high-affinity states of D2 receptors are elevated in dopamine supersensitivity, antipsychotic drugs can block or inhibit dopamine supersensitivity.

It is herein submitted that the short-term use of tracer doses of radioactive (+)PHNO in radioimaging procedures will be of diagnostic and therapeutic importance in dopamine-related disorders. In addition to the high selectivity of (+)PHNO for D2 receptors, the dissociation constant of (+)PHNO for D2 is much lower than that of quinpirole, suggesting that (+)PHNO binds more tightly to D2 and would be less sensitive to endogenous dopamine which tends to interfere with the binding of any ligand to D2.

More specifically, the dissociation constant of [³H](+)PHNO for striatal D2 receptors in the presence of physiological NaCl is 0.56±0.08 nM. The inhibition constant, Ki, for (+)PHNO to inhibit the binding of [³H]spiperone to striatal D2 receptors is 1.2 nM, while that for (−)PHNO is 4,400 nM (P. Seeman et al., Synapse 14: 254-262, 1993). The Ki for (+)PHNO to inhibit the binding of either [³H]raclopride or [³H]domperidone (0.6 nM) to striatal D2 receptors is lower than that to inhibit [³H]spiperone binding (P. Seeman and H. H. M. Van Tol, Eur. J. Pharmacol. 291: 59-66, 1995; P. Seeman et al., Synapse 49: 209-215, 2003).

In addition to these advantageous features for (+)PHNO, the compound has a low affinity for the high-affinity state of the D1 receptor with a dissociation constant of 80 nM (P. Seeman and H. B. Niznik, ISI Atlas of Science, Pharmacology 2: 161-170, 1988), unlike N-propyl-norapomorphine which has the same affinity for D2^{High} and D1^{High}.

In the case of rats sensitized to become supersensitive to amphetamine, the in vitro binding of [³H](+)PHNO, which labels D2^{High}, increased.

The present invention relates to a method for determining an amount of dopamine D2^{high} receptors in a subject comprising determining specific binding of radiolabelled (+)-4-propyl-9-hydroxy-2,3,4a,5,6,10b-hexahydro-4H-naphth[1,2b][1,4]-oxazine HCl ((+)PHNO) in the subject's brain. The presence of radiolabelled (+)PHNO in the subject's brain indicates the presence of dopamine D2^{high} receptors. Further, the specific binding of radiolabelled (+)PHNO in the subject's brain is correlated with the amount of dopamine D2^{high} receptors in that area, such that the greater the specific binding of radiolabelled (+)PHNO, the greater the number of D2^{high} receptors.

The specific binding of radiolabelled (+)PHNO can be determined using any technique known in the art. In an embodiment of the invention, specific binding is the difference in the amount or density of radiolabelled (+)PHNO bound in a specific region of the subject's brain when the radiolabelled (+)PHNO is administered alone and the amount of radiolabelled (+)PHNO bound in that same region when the radiolabelled (+)PHNO is administered contemporaneously with a non-radiolabelled dopamine agonist or dopamine mimetic. The non-radiolabelled dopamine agonist or mimetic should have an affinity or dissociation constant for the dopamine D2 receptor that is similar to [³H](+)PHNO, for example between about 0.4 to about 0.9 nM for the high-affinity state of dopamine D2 receptors, and a permeability across biological membranes that is similar to that for (+)PHNO. Accordingly, in an embodiment of the invention, the specific binding of radiolabelled (+)PHNO is determined by:

(a) administering an effective amount of radiolabelled (+)PHNO to a subject and observing an amount or density of radiolabelled (+)PHNO in the subject's brain;

(b) allowing a suitable amount of time to pass for spontaneous decay of the radiolabelled (+)PHNO administered in (a);

(c) contemporaneously administering an effective amount of radiolabelled (+)PHNO and an effective amount of a suitable non-radiolabelled dopamine agonist or dopamine-mimetic and observing an amount or density of radiolabelled (+)PHNO in the subject's brain; and

(d) determining a difference between the amount or density of radiolabelled (+)PHNO in (a) and the amount or density of radiolabelled (+)PHNO in (c), wherein said difference is the specific binding of radiolabelled (+)PHNO.

A suitable time for the spontaneous decay of radiolabelled (+)PHNO in step (b) will depend on the identity of the radiolabel and its specific half life. A person skilled in the art would readily be able to determine this time. For example, if the radiolabel is ¹¹C, with a half life of 20 minutes, the suitable time for step (b) may be, for example, more than 3 hours, specifically about 4 hours.

The term an “effective amount” of radiolabelled (+)PHNO as used herein is that amount sufficient to effect desired results, including clinical results, and, as such, an “effective amount” depends upon the context in which it is being applied. For example, in the context of administering an “effective amount” of radiolabelled (+)PHNO, an effective amount is, for example, an amount sufficient to achieve labelling and/or quantification of the D2^{high} receptors in the brain of the subject.

The term “dopamine mimetic” refers to any compound that acts like dopamine or causes a release of dopamine.

Suitable non-radiolabelled dopamine agonists or dopamine mimetics include, but are not limited to amphetamine, (+)—PHNO, apomorphine and congeners thereof (for example, N-propyl-norapomorphine) and various aminotetralins, such as dihydroxy-2-dimethyl-aminotetralin. In an embodiment of the present invention, the non-radiolabelled dopamine agonist or dopamine-mimetic is amphetamine, (+)PHNO, apomorphine or N-propyl-norapomorphine. In a further embodiment of the present invention, the non-radiolabelled dopamine agonist is (+)PHNO or apomorphine.

The term “subject” as used herein includes all members of the animal kingdom including human. The subject is suitably a human.

The contemporaneously administered dose of dopamine agonist or dopamine mimetic should be on the order of about 10 to about 50 times the dose of total active drug or active ingredient in the radiolabelled (+)PHNO dose (radiolabelled and non-radiolabelled molecules), thus defining a baseline to determine the number of high-affinity states of D2 in the same brain region.

As used herein, “administered contemporaneously” means that the two substances are administered to a subject such that they are both biologically active in the subject at the same time. In particular embodiments, two substances will be administered substantially simultaneously, i.e. within seconds of each other, or desirably in a single composition that comprises both substances.

In a further embodiment of the present invention, the amount or density of radiolabelled (+)PHNO in steps (a) and (c) is determined in specific regions of the subject's brain. For example, the amount or density of radiolabelled (+)PHNO, may be observed in the striatum, the caudate nucleus, the putamen regions and/or the globus pallidus. The region used in (a) will be the same as that used in (c).

The terms “determining” and “observing” are meant to include both qualitative and quantitative determinations of the amount or density of D2^{high} receptors localized in the brain or in the specified areas of the brain.

The radiolabelled (+)PHNO may be (+)PHNO incorporating any radioactive isotope suitable for nuclear medical imaging, for example suitable for PET or SPECT. This may include analogs of (+)PHNO containing an additional radioactive label, for example [¹⁸F](+)PHNO, yet retaining the same receptor binding profile as (+)PHNO. In an embodiment of the invention, the radiolabelled (+)PHNO is [¹¹C]-(+)-4-propyl-9-hydroxy-2,3,4a,5,6,10b-hexahydro-4H-naphth[1,2b][1,4]-oxazine HCl or [¹¹C]-(+)PHNO. [³H]PHNO is available via tritiation of the allyl precursor of (+)PHNO and is available from commercial sources (for example, Perkin Elmer Life Sciences). [¹¹C]PHNO may be prepared using the method described by Brown et al. (J. Brown, S. K. Luthra, F. Brady, C. Prenant, D. Dijkstra, H. Wikstrom, and D. Brooks, Labelling of the D2 agonist (+)—PHNO using [¹¹C]-Propionyl Chloride, in XIIth Int. Symp. Radiopharmaceutical Chemistry, pp. 565-566, Uppsala, Sweden, 1997) by reaction of (+)-3,4,4a,5,6,10b-hexahydro-2H-naphtho[1,2-b][1,4]oxazin-9-ol, hydrochloride with [¹¹C]-propionyl chloride followed by reduction with for example lithium aluminium hydride.

The amount or density of radiolabelled (+)PHNO in the subject's brain may be determined or observed, using any known technique to detect or image radioactive compounds in vivo. As is known, the presently available nuclear medicine imaging procedures for human use are single photon emission computed tomography, SPECT, and positron emission tomography, PET. In an embodiment of the invention, the specific binding of radiolabelled (+)PHNO in the subject's brain is determined or observed using PET.

In an embodiment of the present invention, the specific binding of radiolabelled (+)PHNO in the brain of the subject is compared to a control and if the specific binding is greater in the subject compared to the control then the subject is in a state of dopamine supersensitivity. As used herein, the term “control” means the specific binding of radiolabelled (+)PHNO that would be in one or more regions of the subject's brain under standard or normal conditions. By “standard” or “normal” conditions it is meant in the absence of disease, injury, medication and/or substance (i.e. drug or alcohol) abuse, or any other factor that would affect the amount of D2^{high} receptors in the brain. For example, a control may be a subject that does not have any psychiatric illness of drug-induced illness. The term “greater” refers to any detectable increase in the specific binding of radiolabelled (+)PHNO in one or more regions of the subject's brain of the subject compared to the control.

Animals of significantly different weight may lead to different distributions in the body of the radioactive dose. If this occurs, it is standard practice to normalize the final values for specific binding by dividing each of the specific binding values by the amount of total DPM/mg found in the cerebellum.

In a further embodiment of the present invention, there is included a method of determining an extent of dopamine supersensitivity in a subject comprising determining specific binding of radiolabelled (+)PHNO in the subject's brain.

Since the presence of radiolabelled (+)PHNO in the subject's brain indicates the presence of dopamine D2^{high} receptors and the specific binding of radiolabelled (+)PHNO in the various regions of the subject's brain is correlated with the amount of dopamine D2^{high} receptors in that area, such that the greater the specific binding of radiolabelled (+)PHNO, the greater the number of D2^{high} receptors, the method of the present invention can be used to determine if a subject is in a state of dopamine supersensitivity. The extent of dopamine supersensitivity is an important factor in the assessment of health and disease in a subject, for example, to assess, treat and/or follow the progress of any dopamine-related disorder. If a subject has elevated levels of dopamine D2^{high} receptors in their brain compared to a control, then they may be considered to have dopamine supersensitivity. By elevated levels, it is meant that the levels or amount of D2^{high} receptors in the subject are greater than that in a control, as defined above. Such supersensitivity affects their reaction to dopamine related drugs, for example dopamine agonists, and is a significant consideration in the diagnosis and course of treatment for the subject.

The term “dopamine-related disorder” as used herein refers to any disorder, disease or condition which is the result of modulation of, or causes a modulation in, the activity at a dopamine receptor, in particular the D2^{High} receptors. In embodiments of the invention, the dopamine-related disorder is selected from Parkinson's disease, psychoses, schizophrenia, addiction, attention-deficit hyperactivity disorder (ADHD or ADD), adult attention-deficit disorder (AADD), depression, Huntington's disease and Progressive Supranuclear Palsy.

As a representative, non-limiting example, whether or not a Parkinson diseased subject is or is not sensitive to treatment with L-DOPA, or some other dopamine agonist, may depend on the number of high-affinity states of D2 that exist in that particular subject. Likewise, similar determinations are critical in the treatment and diagnosis of psychoses and schizophrenia. In a further aspect of the present invention, the specific binding of radiolabelled (+)PHNO is determined in the globus pallidus and this amount is used to diagnose early stages of Progressive Supranuclear Palsy (PSP). Accordingly, the present invention also includes a method of diagnosing PSP in a subject comprising observing specific binding of radiolabelled (+)PHNO in the globus pallidus of the subject. In an embodiment of the invention the specific binding of radiolabelled (+)PHNO in the globus pallidus of the subject is compared to a control and if there is an alteration or dimunition in the amount or pattern of the specific binding of radiolabelled (+)PHNO in the globus pallidus in the subject compared to the control, then the subject may have early stage PSP.

In a further embodiment of the present invention, the amount of D2^{high} receptors in a subject's brain is expressed as a percentage of the total population of dopamine D2 receptors. This is done by dividing the specific binding of radiolabelled (+)PHNO by the total D2 density and multiplying by 100. The total D2 density may be determined in humans or animals using methods known to those skilled in the art, for example using [¹¹C]raclopride or [³H]raclopride (A. Abi-Dargham, et al., Proc. Nat. Acad. Sci., U.S.A., 72:7673, 2000).

The present invention also includes the use of radiolabelled (+)PHNO to determine an amount dopamine D2^{high} receptors in a subject as well as the use of radiolabelled (+)PHNO to prepare a medicament to determine an amount dopamine D2^{high} receptors in a subject.

Further, the present invention includes the use of radiolabelled (+)PHNO to determine an extent of dopamine supersensitivity in a subject as well as the use of radiolabelled (+)PHNO to prepare a medicament to determine an extent of dopamine supersensitivity in a subject.

The radiolabelled (+)PHNO is preferably formulated into pharmaceutical compositions for administration to human subjects in a biologically compatible form suitable for administration in vivo. Accordingly, in another aspect, the present invention provides a pharmaceutical composition comprising radiolabelled (+)PHNO in admixture with a suitable diluent or carrier.

In accordance with the methods of the invention, the radiolabelled (+)PHNO may be administered to a patient in a variety of forms depending on the selected route of administration, as will be understood by those skilled in the art. The compositions of the invention may be administered, for example, by intraveneous administration and the radiopharmaceutical compositions formulated accordingly, for example together with any physiologically and radiologically tolerable vehicle appropriate for administering the compound systemically.

In an embodiment of the invention, the compounds are administered intravenously to minimize metabolism before the compound enters the brain. The amount or dosage of radiolabelled (+)PHNO required to image or quantify the D2^{high} in the brain will be readily ascertained by one of ordinary skill in the nuclear medicine art taking into account the specific activity of the compound and the radiation dosimetry. As is known by those skilled in the nuclear medicine art, the number of milliCuries of the radiolabelled compound to be administered for the PET or SPECT scan will be limited by the dosimetry, whereas the mass of compound to be administered (e.g. μg/kg or mg/kg of body weight of the patient) is calculated based on the specific activity of the synthesized compound, i.e., the amount of radioactivity/mass, of radiolabelled compound. It will be appreciated that because of the short half-life of the radioisotopes, e.g. about 20 minutes for ¹¹C, it is often necessary to make the radiolabelled compound at or near the site of administration. The specific activity of the compounds must then be ascertained in order to calculate the proper dosing. Such techniques are well known to those skilled in the art.

By way of illustration, and not in limitation, the amount of radiolabelled (+)PHNO to be administered to a human subject is a minimum of 10 milliCuries (mCi) of radiolabelled (+)PHNO, administered intravenously. The amount of radiolabelled (+)PHNO to be administered to a rat may be a minimum of 0.5 mCi. The maximum amount of radiolabelled (+)PHNO would be that amount that would be harmful or toxic to the subject. In an embodiment of the invention the radiolabelled (+)PHNO is administered using a bolus infusion protocol (R. E. Carson et al., J. Cereb. Blood Flow Metab. 17: 437-447, 1997), with 60% of the dose injected as a bolus over 1 min and the rest injected by means of intravenous infusion over 75 min.

In a further embodiment of the present invention. the radiolabelled (+)PHNO is used in in vitro screens for compounds that bind to the D2^{High} receptors. D2^{High} receptor ligand candidates may be identified by first incubating a sample comprising the D2^{High} receptor with radiolabelled (+)PHNO then incubating the resulting preparation in the presence of the candidate ligand. A more potent D2^{High} receptor ligand will, at equimolar concentration, displace the radiolabelled (+)PHNO. Accordingly, the present invention includes a method of screening for compounds that bind to the D2^{High} receptor comprising (a) combining a sample comprising the D2^{High} receptor with radiolabelled (+)PHNO and test compound under conditions sufficient for binding of radiolabelled (+)PHNO and the test compound to the D2^{High} receptor; and (b) determining an amount of binding of the radiolabelled (+)PHNO that is inhibited in the presence of the test compound, wherein the greater the amount of binding of radiolabelled (+)PHNO that is inhibited in the presence of the test compound, the greater the binding of the test compound to the D2^{High} receptors.

The invention also includes the use of radiolabelled (+)PHNO to screen for compounds that bind to the D2^{High} receptors.

The radiolabelled (+)PHNO may be (+)PHNO incorporating any radioactive isotope suitable for standard radiolabel detection using, for example, scintillation spectrometers. This may include analogs of (+)PHNO containing an additional radioactive label, for example [¹⁸F](+)PHNO, yet retaining the same receptor binding profile as (+)PHNO. In an embodiment of the invention, the radiolabelled (+)PHNO is [¹¹C]-(+)-4-propyl-9-hydroxy-2,3,4a,5,6,10b-hexahydro-4H-naphth[1,2b][1,4]-oxazine HCl or [¹¹C]-(+)PHNO.

The sample comprising the D2^{High} receptor may be, for example, any preparation containing cells that express this receptor. The cells may be cells that naturally contain the D2^{High} receptor or may be cells that have been transformed specifically to produce the D2^{High} receptor. Such cell lines are well known in the art.

The test compound may be any compound that one wishes to test for binding to the D2^{High} receptor and it may be a mixture of compounds, for example, from a combinatorial library.

The following non-limiting examples are illustrative of the present invention:

### EXAMPLES

### Example 1

### In Vivo Determination of Specific Binding in Humans

For humans, a minimum amount of 10 milliCuries (0.5 mCi for rats) of radioactive (+)PHNO, for example [¹¹C](+)PHNO (500-1,000 mCi/μmole) is injected intravenously in a human volunteer, using a bolus plus infusion protocol (R. E. Carson et al., J. Cereb. Blood Flow Metab. 17: 437-447, 1997), with 60% of the dose injected as a bolus over 1 min and the rest injected by means of intravenous infusion over 75 min.

Positron emission scans are done every 60 seconds for 15 min, and then every 300 seconds until the study is completed at one hour and fifteen minutes. The scanning is done by a brain scanner that produces 6.5 mm-wide slices with a resolution of at least 5 mm.

The regions of interest to be measured include the putamen, the head of the caudate nucleus and the globus pallidus. The peak emission in the caudate nucleus occurs at 10 minutes after the intravenous injection of the order of 0.2% of the injected dose per kg. Several hours after the initial injection of radioactive (+)PHNO, a second injection of radioactive (+)PHNO is given intravenously at the same time as a very low dose of NPA (i.e., N-propyl-norapomorphine) or another suitable dopamine agonist or dopamine-mimetic such as amphetamine. The co-administered dose of radioactive (+)PHNO or NPA (i.e., N-propyl-norapomorphine should be on the order of about 10 to about 50 times the dose of total drug in the radioactive (+)PHNO dose (radiolabelled and non-radiolabelled molecules), thus defining a baseline to determine the number of high affinity states of D2 in the same brain region. The difference between the brain image done without co-injection and the image with co-injection is defined as specific binding, and indicates the presence of D2^{high} receptors. The density of the D2^{High} sites in the caudate nucleus that are labelled by radioactive (+)PHNO is calculated, knowing the specific activity of the tracer injected and the amount of radioactivity detected by the positron imaging camera.

To confirm that the pattern of radioactive (+)PHNO specific binding to brain tissue truly reflects binding to dopamine D2 receptors, the following experiments are done: A. The effect of reserpine and/or alpha-methyl-paratyrosine is to increase the specific binding, because endogenous dopamine is removed. B. The amount of specific binding is to be found highest in the striatum (caudate nucleus and putamen), with low amount of nonspecific binding in all the other brains regions, including the cerebellum. C. Antipsychotic drugs given prior to the injection of radioactive (+)PHNO reduce the magnitude of specific binding. D. Non-dopaminergic drugs do not affect the specific binding of radioactive (+)PHNO.

### Example 2

### Validation of Method in Rats

In describing and validating the method of the present invention using rats instead of humans, there are certain considerations that should be noted. For example, each rat is not imaged, but is guillotined, and the striatum removed in order to measure the radioactivity in DPM/mg tissue, where DPM represents disintegrations per minute.

The method of the invention, wherein the number of dopamine D2^{High} receptors are measured in a particular brain region, is detailed in the following steps. Specific considerations are noted in the case of describing the principle of the method when using rats.

### (a) Measurement of Total Binding of Radioactive (+)PHNO in the Striatum (Striatum DPM/mg):

The first step is to inject radioactive (+)PHNO and determine the total amount of radioactive (+)PHNO that becomes located in a specific region at a specific moment in time. In the case of humans, however, those trained in the art are recommended to determine the binding potential of a brain region (by the customary method) from the complete time-course of the binding brain scan when using radioactive (+)PHNO. The binding potential is defined as Bmax/Kd, where Bmax is the density of D2 receptors and Kd is the dissociation constant of the radioactive (+)PHNO.

In the case of rats, the most suitable time for observing the binding of radioactive (+)PHNO is 30 minutes after the injection of radioactive (+)PHNO, at which time the rat is guillotined, and the striata are removed.

Sprague-Dawley rats (275-300 g) were restrained in transparent polystyrene cages. The tail vein was warmed in warm water. One ml of saline containing 0.1 nmol radioactive (+)PHNO ([³H](+)PHNO; PerkinElmer Life Sciences, 50 Ci/mmol; 5 μL in 1 ml saline), was injected into the pre-warmed tail vein. Thirty minutes after the injection of the radioactive (+)PHNO ([³H](+)PHNO; PerkinElmer Life Sciences, 50 Ci/mmol; 5 μL in 1 ml saline) the average total binding of radioactive (+)PHNO was 33.9 DPM per mg of striatum (n=6). This number can be converted to pmols of D2 receptors per mg of tissue by using the specific activity of radioactive (+)PHNO.

A second experiment was done using contemporaneous injection of a dose of NPA (a dopamine agonist) simultaneously with the same dose of radioactive (+)PHNO that was used in step 1 above. The injected NPA competes with the injected radioactive (+)PHNO and inhibits the binding of the injected radioactive (+)PHNO to the D2^{High} receptors. Accordingly, an injection containing NPA (25 μg/Kg) and 0.1 nmol of radioactive (+)PHNO ([³H](+)PHNO; PerkinElmer Life Sciences, 50 Ci/mmol; 5 μL in 1 ml saline) was made into the tail veins of Sprague_Dawley rats (300 g per rat).

In the case of the rats, an injection of 25 μg/kg NPA (or N-propyl-norapomorphine) contemporaneously injected with the dose of radioactive (+)PHNO used in step 1 above, the binding of radioactive (+)PHNO to the D2 receptors was 16.02 DPM/mg striatum.

In other words, the binding of radioactive (+)PHNO went from a total of 33.9 DPM/mg striatum down to 16.02 DPM/mg striatum, a fall of about 17.9 DPM/mg or about 53% (n=6). This indicates that the standard dose of 25 μg/kg of NPA inhibited 53% of the D2^{High} receptors in these normal rats.

When using radioactive (+)PHNO in the case of humans, the binding potential of the striatal region (or other brain region of interest) monitored in this second experiment (or second measurement) is subtracted from the binding potential in the first measurement specified above. The difference between these two measures is an indication of the “specific D2^{High} binding” in the region of interest.

In order to measure D2^{High} receptors in dopamine-supersensitive conditions, the same steps given above may be done. Should the number of D2^{High} receptors increase in the supersensitive condition, for example, the effect of the contemporaneously injected dopamine agonist will be greater than the inhibitory effect demonstrated above in the control humans or animals.

In the case of humans, the co-injection of a dopamine agonist may cause some nausea or possible risk of vomiting, because dopamine agonists stimulate D2 receptors in the vomiting center of the area postrema in the brainstem. To obviate this risk, it may be necessary to pre-medicate the volunteer with a low dose of domperidone, which is a dopamine D2 blocking compound that does not permeate into the brain itself. Domperidone will only enter the area postrema to block vomiting, because the area postrema does not have a BBB (blood-brain barrier).

### Example 3

### Comparison with NPA and Radioactive Raclopride in Rats

In order to demonstrate that the D2 receptors labeled by radioactive (+)PHNO are different than those labeled by radioactive raclopride, the steps described in Example 2 were repeated in rats using 0.1 nanomole radioactive raclopride ([³H]raclopride; PerkinElmer Life Sciences, 62.2 Ci/mmol; 6.2 μL in 1 ml saline).

It was found that the inhibition of the binding of radioactive raclopride by the contemporaneous intravenous co-injection of NPA (25 μg/Kg) was not identical and was smaller than the inhibition of radioactive (+)PHNO by NPA.

In fact, in a representative experiment, it was found that 25 μg/Kg NPA inhibited the binding of radioactive (+)PHNO by 54% to a level of 46% (mean ±s.e.; n=6) of the control value, while only inhibiting the binding of radioactive raclopride by 39% to a level of 61%±2.9% (mean ±s.e.; n=6) of the control value (P=0.0004). The actual data are shown in Table 1.

The finding that the radioactive agonist (+)PHNO ligand is more sensitive than radioactive raclopride to inhibition by the NPA agonist indicates that the two ligands are measuring different populations of D2 receptors.

These results for the co-injection method are in contrast to the pre-injection method used by McCormick et al. (McCormick P N, Kapur S, Seeman P, Rabiner E A, Wilson A A. 2007. Dopamine D2 receptor radiotracers, [¹¹C]-(+)-PHNO and [³H]-raclopride, are indistinguishably inhibited by D2 agonists and antagonists ex vivo. Nucl Med Biol (in press)) where NPA was injected subcutaneously 30 minutes prior to the injection of the radioligand.

This experimental observation, therefore, confirms that radioactive (+)PHNO labels D2^{High} receptors that are different than the population of D2 receptors being labeled by radioactive raclopride.

The major biological difference between the pre-injection method (McCormick et al., 2007. Id) and the method of the present invention (the co-injection methods) is that the co-injection method ensures that the arrival of the radioligand and the cold agonist occur at precisely the same moment. Considering that a receptor agonist can rapidly desensitize a receptor within seconds, the arrival of cold agonist prior to that of radioactive (+)PHNO would reduce the amount of D2^{High} receptors available to radioactive (+)PHNO. Therefore, in contrast to McCormick et al. (2007), the present data confirm the existence of high- and low-affinity states of the dopamine D2 receptor in vivo.

These results confirm that the method of the present invention is recommended for objectively measuring D2^{High} in relation to the development, the diagnosis, and the treatment of dopamine supersensitivity in various disease states.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

TABLE 1

| | | | | | pmol/g |
|---|---|---|---|---|---|
| | Control | | With NPA | | Displaced by |
| Ligand | DPM/mg | pmol/g | DPM/mg | pmol/g | NPA |
| [³H](+)PHNO | 33.9 | 3.12 | 16.02 | 1.47 | 1.65 |
| [³H]Raclopride | 37.9 | 2.8 | 23.13 | 1.71 | 1.09 |

What is claimed is:

1. A method for determining an amount of dopamine D2^{high} receptors in a subject comprising determining specific binding of radiolabelled (+)-4-propyl-9-hydroxy-2,3,4a,5,6,10b-hexahydro-4H-naphth[1,2b][1,4]-oxazine HCl ((+)PHNO) in the subject's brain, wherein the specific binding of radiolabelled (+)PHNO is correlated with the amount of dopamine D2^{high} receptors in the subject's brain and is the difference between an amount or density of radiolabelled (+)PHNO in the subject's brain obtained when the radiolabelled (+)PHNO is administered without contemporaneous administration of non-radiolabelled dopamine agonist or mimetic and an amount or density of radiolabelled (+)PHNO in the subject's brain obtained with contemporaneous injection of non-radioactive dopamine agonist or mimetic.

2. The method according to claim 1, wherein the subject is human.

3. The method according to claim 1, wherein the specific binding of radiolabelled (+)PHNO in the subject's brain is observed using PET.

4. The method according to claim 1, wherein the specific binding of radiolabelled (+)PHNO is observed in a region of the brain selected from one or more of the striatum, caudate nucleus, putamen and globus pallidus.

5. The method according to claim 1, wherein the radiolabelled (+)PHNO is [¹¹C]-(+)-4-propyl-9-hydroxy-2,3,4a,5,6,10b-hexahydro-4H-naphth[1,2b][1,4]-oxazine HCl ([¹¹C]-(+)PHNO).

6. The method according to claim 1, wherein the amount of D2^{High} receptors in the subject's brain is used to assess, treat and/or follow the progress of any dopamine-related disorder.

7. The method according to claim 6, wherein the dopamine-related disorder is selected from psychoses, schizophrenia, Parkinson's disease, Progressive Supranuclear Palsy, addiction, attention-deficit hyperactivity disorder (ADHD or ADD), adult attention-deficit disorder (AADD) and depression.

8. The method according to claim 1, further comprising correlating the amount of dopamine D2^{high} receptors with an extent of dopamine supersensitivity in the subject.

9. The method according to claim 1, wherein the specific binding of radiolabelled (+)PHNO in the brain of the subject is compared to a control and if the specific binding is greater in the subject compared to the control then the subject is in a state of dopamine supersensitivity.

10. The method according to claim 8, wherein the extent of dopamine supersensitivity is used to assess, treat and/or follow the progress of any dopamine-related disorder.

11. The method according to claim 10, wherein the dopamine-related disorder is psychoses, schizophrenia, addiction, ADHD, AADD depression, Huntington's Disease, Progressive Supranuclear Palsy or Parkinson's disease.

12. The method according to claim 1, wherein the specific binding of (+)PHNO is determined by:
(a) administering an effective amount of radiolabelled (+)PHNO to a subject and observing an amount or density of radiolabelled (+)PHNO in the subject's brain;
(b) allowing a suitable amount of time to pass for spontaneous decay of the radiolabelled (+)PHNO administered in (a);
(c) contemporaneously administering an effective amount of radiolabelled (+)PHNO and an effective amount of a suitable non-radiolabelled dopamine agonist or dopamine mimetic and observing an amount or density of radiolabelled (+)PHNO in the subject's brain; and
(d) determining a difference between the amount or density of radiolabelled (+)PHNO in (a) and the amount or density of radiolabelled (+)PHNO in (c), wherein said difference is the specific binding of radiolabelled (+)PHNO.

13. The method according to claim 12, wherein the radiolabelled (+)PHNO and non-radiolabelled dopamine agonist or dopamine mimetic are formulated into radiopharmaceutical compositions for administration to human subjects in a biologically compatible form suitable for administration in vivo.

14. The method according to claim 13, wherein the compositions are administered by intraveneous administration.

15. The method according to claim 13, wherein the radiopharmaceutical compositions are formulated together with any physiologically and radiologically tolerable vehicle appropriate for administering the compound systemically.

16. The method according to claim 1, wherein the specific binding of radiolabelled (+)PHNO is divided by a total density of dopamine D2 receptors to provide a percentage of dopamine D2^{high} receptors in the subject's brain.

17. The method according to claim 16, wherein the percentage of dopamine D2^{high} receptors in the subject's brain is compared to a control and if the percentage of dopamine D2^{high} receptors is greater in the subject compared to the control then the subject is in a state of dopamine supersensitivity

18. A method of screening for compounds that bind to the D2^{High} receptor comprising (a) combining a sample comprising the D2^{High} receptor with radiolabelled (+)PHNO and test compound under conditions sufficient for binding of radiolabelled (+)PHNO and the test compound to the D2^{High} receptor; and (b) determining an amount of binding of the radiolabelled (+)PHNO that is inhibited in the presence of the test compound, wherein the greater the amount of binding of radiolabelled (+)PHNO that is inhibited in the presence of the test compound, the greater the binding of the test compound to the D2^{High} receptors.
